(19) United States
(12) Patent Application Publication
(10) Pub. No.: US 2015/0367031 A1
(43) Pub. Date: Dec. 24, 2015

(54) METHODS AND COMPOSITIONS FOR INHIBITING FIBROSIS, SCARRING AND/OR FIBROTIC CONTRACTURES

(71) Applicant: WAKE FOREST UNIVERSITY HEALTH SCIENCES, Winston-Salem, NC (US)

(72) Inventors: Louis Argenta, Winston-Salem, NC (US); Nicole H. Levi, Winston-Salem, NC (US); Michael Morykwas, Winston-Salem, NC (US); Elizabeth Walles, Winston-Salem, NC (US); Maria McGee, Winston-Salem, NC (US); William D. Wagner, Clemmons, NC (US); Elizabeth Graham, Mount Pleasant, SC (US)

(21) Appl. No.: 14/765,734

(22) PCT Filed: Apr. 2, 2014
(86) PCT No.: PCT/US14/32673

§ 371 Date: Aug. 4, 2015

Related U.S. Application Data
(60) Provisional application No. 61/807,503, filed on Apr. 2, 2013.

(51) Int. Cl. A61L 27/34; A61L 27/56; C01B 31/02; A61L 27/54

(52) U.S. Cl. CPC A61L 27/34; A61L 27/54; A61L 27/56; C01B 31/022; A61L 2430/04; A61L 2420/06; A61L 2300/606; A61L 2300/108; C01B 2202/06

(57) ABSTRACT

Biomedical implants, regenerative scaffolds, and compositions comprise a substrate with a coating, a scaffold, and/or a carrier composition which include anisotropic nanoparticles in or on the coating, scaffold or carrier, to inhibit fibrosis, scarring, and/or fibrotic contracture, or the formation of adhesions, in a tissue contacting or administered the same. In some embodiments the nanoparticles may be electrically conductive nanoparticles such as multi wall carbon nanotubes.

### RELATED APPLICATIONS

This application claims the benefit of U.S. Provisional Patent Application Ser. No. 61/807,503, filed Apr. 2, 2013, the disclosure of which is incorporated by reference herein in its entirety.

### BACKGROUND OF THE INVENTION

Fibroblasts are the main connective tissue type in the body and maintain the stroma for numerous other cell types including keratinocytes, myocytes, and alveolar epithelial cells.^{[1-3]} When the tissue is injured, these cells are the main repair mechanism to repopulate the cells lost to injury, build new extracellular matrix (ECM) and contract it to match the new matrix with undamaged tissue.^{[4]} Sometimes this process causes over-contraction, which leads to both a loss of tissue structure and functionality.^{[5-6]}

The ECM is predominantly composed of type I collagen, but also contains type III collagen, fibronectin, elastin, proteoglycans and glycoproteins.^{[7]} There is a resting mechanical tension in the matrix which shields cells from the shear stresses of everyday motion, but the absence of a matrix in wounded tissue transfers this tension to the cells.^{[8-9]} Mesenchymal cells have been shown to differentiate into fibroblasts and myofibroblasts to heal acute and chronic wounds.^{[11]} Specifically, fibroblasts normally remodel the collagen, but they cannot generate much force to contract the ECM.^{[12]} To close the wound, fibroblasts differentiate into myofibroblasts, which secrete a greater volume of ECM proteins than regular fibroblasts and also express α-smooth muscle actin to better compact the new matrix.^{[13]} Once the cells have restored the resting tension in the matrix, the myofibroblasts usually apoptose in large numbers.^{[14]} However, sometimes the cells do not disengage. These cells continue to contract forcefully and deposit excessive collagen without organization such as extensive crosslinking or bundle formation.^{[2, 15]} This pathology results in a number of fibrotic diseases in the skin, heart, lungs, liver, kidneys and the stroma reaction to epithelial tumors which aids tumor growth and metastasis.^{[14, 16-19]}

### SUMMARY OF THE INVENTION

A first aspect of the present invention is a biomedical implant, comprising:

(a) an inert substrate (e.g., a flexible, rigid, or semirigid substrate);

(b) a coating on said substrate; and

(c) anisotropic nanoparticles in or on said coating in an amount effective to inhibit fibrosis, scarring, and/or fibrotic contracture of a tissue contacting said implant when implanted adjacent said tissue in a subject in need thereof.

A second aspect of the invention is a method of inhibiting fibrosis, scarring, and/or fibrotic contracture of tissue contacting a biomedical implant in a patient implanted with said biomedical implant, comprising: administering anisotropic nanoparticles to said tissue in an amount effective to inhibit fibrosis, scarring, and/or fibrotic contracture of said tissue.

Also described herein is a regenerative template for implantation in a subject, comprising:

(a) a porous tissue scaffold; and

(b) anisotropic nanoparticles coated on said scaffold in an amount effective to inhibit fibrosis, scarring, and/or fibrotic contracture of tissue contacting said template when implanted in a subject in need thereof.

A further aspect of the invention is a method of inhibiting fibrosis, scarring, and/or fibrotic contracture of tissue contacting or infiltrating a regenerative template in a patient implanted with said regenerative template, comprising: administering anisotropic nanoparticles to said tissue in an amount effective to inhibit fibrosis, scarring, and/or fibrotic contracture of said tissue.

Also described herein is a method of inhibiting the formation of tissue adhesions in a subject in need thereof, comprising topically administering anisotropic nanoparticles to said tissue in an effective adhesion-inhibiting amount.

Also described herein is a composition useful for inhibiting the formation of tissue adhesions in a subject in need thereof, comprising:

(a) a pharmaceutically acceptable carrier; and

(b) anisotropic nanoparticles in said carrier.

In some embodiments of implants, scaffolds, and compositions of the foregoing, the coating or scaffold comprises a water binding polymer, or the carrier contains a water binding polymer.

In some embodiments of the methods of the foregoing, the methods further comprise concurrently administering a water binding polymer to said tissue in an amount effective to inhibit fibrosis, scarring, and/or fibrotic contracture of the tissue, or in an effective adhesion-inhibiting amount

Objects of the present invention are to provide treatments for the foregoing conditions that do not substantially impede function of the subject organs or conditions, and/or promote the formation of substantially native tissue architecture.

E. Unger et al., US Patent Application Publication No. 20040247624 (Dec. 9, 2004) describes drug formulations carried by anisotropic nanoparticles. The anistropic structure of the particles is suggested to aid in laminar flow after vascular administration and “flipping” of the nanoparticles to lodge them in tissue by ultrasound (see paragraph 0215 therein). Use of the nanoparticles in inhibiting fibrosis, scar formation or fibrotic contractures is neither suggested nor described.

The present invention is explained in greater detail in the specification set forth below. The disclosures of all United States patent references cited herein are to be incorporated herein by reference in their entirety.

### BRIEF DESCRIPTION OF THE DRAWINGS

FIG. 1: Gels of each material type at each concentration tested. The 0% gel is the positive control, which contains no nanoparticles. Gels are shown on the same day that they were made (day 0 contraction). Red/purple coloration is due to phenol red in the culture media.

FIG. 2: Multi-walled carbon nanotubes (MWNT) and single-walled carbon nanotubes (SWNT) decrease gel contraction at every concentration tested. The area of each gel type at day 7 was normalized by the area of a non-cellular but otherwise identical gel to evaluate the extent of cell-mediated contraction. An asterisk denotes significance from the control on the p<0.05 level. Error bars show the standard deviation of the normalized data.

FIG. 3: MWNT and SWNT increase cell viability only at the highest concentration tested, while carbon black increases cell viability only at the intermediate concentration. Cell counts were generated by dissolving each gel in collagenase, pipetting to homogenize the solution, counting an aliquot of the solution using a hemocytometer, and then measuring the total volume of solution. Counts shown are the product of the concentration generated using the hemocytometer and the total volume of solution. An asterisk denotes significance from the control on the p<0.05 level. A dagger denotes a significant difference between different concentrations of the same material. Error bars show the standard deviation.

FIG. 4: A gel preparation method described herein creates a uniform distribution of the nanoparticles throughout the gel. Each nanoparticle gel contains the maximum concentration tested (1%). Polarized light microscopy of a representative gel of each material are shown at day 7. No aggregates are present in any gel type. All gels are shown at 10× magnification.

FIGS. 5A-5B: Live and dead cell populations were evaluated on day 3, and demonstrate a similar increase in cell viability as was determined using the MTS cell viability, at day 7, as shown in FIG. 3.

FIG. 5A. Confocal images of gels stained with a Live/Dead assay after 3 days of incubation. Live cells are green and dead cells are red.

FIG. 5B. Cell counts of each image shown in part A.

FIGS. 6A-6B: Blocking proliferation with Ara-C prevents the disparities observed between different gel types at day 7. Error bars show the standard deviation.

FIG. 6A. The counts measure the total number of viable cells recovered from gels that were dissolved after 7 days of incubation and counted using a hemocytometer identically to the data collection process described in FIG. 3. No treatment differs from the control. Daggers indicate a significant difference on a p<0.05 level between nanoparticle gel types.

FIG. 6B. Inhibiting proliferation of the cells removes the statistical differences observed in gel contraction. Gel area after 7 days of incubation was normalized by the area of the control gel.

FIGS. 7A-7B: All three actin isoforms are expressed in each gel type with no significant difference in actin expression between materials.

FIG. 7A. The presence of a smooth muscle actin as stained with the anti-ACTA2 antibody further proves the myofibroblastic differentiation of the cells. All gels are viewed at 40× magnification.

FIG. 7B. No statistical difference was observed between any of the groups due to the large variability of the data. Error bars show the standard error of the mean.

FIGS. 8A-8B: MWNT have an antioxidant capability on par with superoxide dismutase, while carbon black is no different from the control and SWNT have too much variability to be accurately described.

FIG. 8A. The temporal response of each gel type to a reactive oxygen species assay.

FIG. 8B. The endpoints of each gel type after 90 minutes of the assay. Error bars are the standard error of the mean and asterisks denote significance on a p<0.05 level.

FIG. 9: None of the gel types statistically differ in stiffness from the control. However, nanoparticle inclusion in the gels results in altered mechanical properties within each group of nanotube gels. Daggers indicate the differences between various concentrations of the same gel type. Error bars show the standard error of the mean.

### DETAILED DESCRIPTION OF THE INVENTION

Subjects that may be treated by the methods, compositions and materials of the present invention include both human subjects, and animal (typically mammalian) subjects such as dogs, cats, horses, cattle, etc., for veterinary purposes. The subjects may be male or female and of any suitable age, including neonate, infant, juvenile, adolescent, adult, and geriatric subjects. In some embodiments the subjects are those who have been administered radiotherapy or chemotherapy, or who are concurrently undergoing radiotherapy or chemotherapy (e.g., simultaneously with or shortly after receiving a biomedical implant such as for reconstructive purposes after surgery) (see, e.g., U.S. Pat. Nos. 8,388,971; 8,268,888; 8,114,885; 7,972,609), as such treatments may exacerbate undesired scar formation in the subject.

“Carrier” as used herein refers to a diluent, excipient, or vehicle with which nanoparticles are administered. Carriers for topical administration are preferred, including liquid, gel, gas (spray) and combinations thereof (e.g., sprays of liquid particles carrying nanoparticles, which liquid particles are carried by a gas or propellant). Pharmaceutically acceptable carrier” generally refers to a carrier that is safe, non-toxic and neither biologically nor otherwise undesirable, and includes a carrier that is acceptable for veterinary use as well as human pharmaceutical use. A “pharmaceutically acceptable carrier” as used in the present application includes both one and more than one such carrier. Numerous such carriers are known. See, e.g., U.S. Pat. Nos. 8,106,209; 7,851,474; 7,799,337; etc.

“Water binding polymer” as used herein includes organic polymers, which may be neutral polymers, examples of which include but are not limited to proteins and peptides such as albumin (e.g., human serum albumin, bovine serum albumin, etc.), polysaccharides such as dextran, polyethers such as polyethylene glycol, etc., at any suitable molecular weight (e.g., 200, 400 or 800 daltons to 40,000 or 50,000 daltons).

### 1. Nanoparticles.

Nanoparticles for carrying out the present invention may be in any shape and include rods, ellipsoids, spheroids, tubes (single walled and multi-walled), and complex or combined shapes (e.g., as demonstrated by S. Chen, Z. L. Wang, J. Ballato, S. Foulger, and D. L. Carroll, “Monopod, Bipod, and Tetrapod Gold Nanocrystals”, Journal of the American Chemical Society ja038927. December (2003)). The nanoparticles may be composed of any suitable material including carbon (doped and undoped) metals (such as silver, gold, zinc, copper, platinum, iridium, tantalum, etc., including alloys thereof), ceramic (silicon, silica, alumina, calcite, hydroxyapatite, etc.) organic polymers (including stable polymers and bioabsorbable polymers), and composites and mixtures thereof, See, e.g., U.S. Pat. Nos. 6,942,897; 6,929,675; 6,913,825; 6,899,947; 6,888,862; 6,878,445; 6,838,486; 6,294,401; etc. The nanoparticles may be conductive (e.g., conductors of electrons), semiconductive, or nonconductive (insulating). The nanoparticles may be metal nanoparticles formed from metals such as silver, copper, gold, platinum, iridium, and alloys thereof. Carbon nanoparticles (e.g., fullerenes) include nanotubes (including both single-wall and multi-wall nanotubes), buckyballs, fullerenes of other configuration (e.g., ellipsoid), and combinations or mixtures thereof. The nanoparticles may be coupled to (e.g., covalently coupled to) other agents (e.g., proteins, peptides, antibodies) or ligands (e.g., to cell-surface proteins or peptides on the cells being delivered) depending upon the particular application thereof

“Anisotropic nanoparticles” for carrying out the present invention may be of any suitable composition such as described above, and refers to nanoparticles that are generally nonspherical in shape. See, e.g., U.S. Pat. No. 7,119,161. Examples of anisotropic nanoparticles include but are not limited to generally one-dimensional nanoparticles such as nanorods, nanowires, nanotubes, etc.; generally two-dimensional nanoparticles such as triangles, plates, sheets, ribbons, etc.; and generally three dimensional nanoparticles such as pyramids, stars, flowers, multi-pods, nanourchins, tadpoles, nanocages, nanorice, nanocorns, nanoboxes, nanocubes, triangular nanoframes, nanodumbbells, platelets, etc. See generally P. Sajanlal et al., Anisotropic nanomaterials: structure, growth, assembly, and functions, NanoReviews 2011 (Open Access); see also E. Hao et al., E. Hao et al., Synthesis and Optical Properties of Anisotropic Metal Nanoparticles, Journal of Fluoroscience 14, 331-341 (2004); C. Murphy et al., Anisotropic Metal Nanoparticles: Synthesis, Assembly, and Optical Applications, J. Phys. Chem. B 109, 13857-13870 (2005).

The nanoparticles may be any suitable size, but generally have average diameters of from 0.5, 1, or 5 nanometers, up to 500, 1000, or 2000 nanometers. The nanoparticles may be fully dispersed from one another, or may form aggregates, though aggregates are preferably not greater than 10 microns in size. The nanoparticles may have a length dimension greater than the average diameter, with lengths up to 5 or 10 microns.

As noted above, the nanoparticles are preferably anisotropic (that is, have a plurality of discrete points formed on the surface thereof), examples including particles having from 2 points up to 20, 24, or 30 points (or a “degree of anisotropy” of from 2 to 20, 24 or 30).

The nanoparticles may be characterized by a high aspect ratio, such as a ratio of average diameter to maximum length dimension of at least 1:2, at least 1:5, up to an aspect ratio of 1:500 or more.

Conductive nanoparticles (e.g., conductors of electrons) are in some embodiments preferred for carrying out the present invention. Such conductive nanoparticles may be formed of or comprise any suitable conductive material, including conductive metals, conductive polymers, conductive carbon or graphite materials, etc.

In some embodiments, the nanoparticles comprise a structural material or composition that imparts a shape thereto, and further comprise one or more active pharmaceutical agents (e.g., agents that themselves promote or cause a biological response such as wound healing, vascularization, etc.). However, because it is the shape (and in some embodiments conductivity) of the nanoparticles that produce a biological response in the present invention, such active agents in some embodiments may be excluded, providing nanoparticles that are essentially free of active pharmaceutical agents, and which consist essentially of the structural material or composition (e.g., metal, carbon, polymer, or composite thereof) from which they are formed.

In some embodiments, the nanoparticles contain or further comprise one more active agents, such as an antifibrotic or anti-scarring active agent, and/or promotes wound healing, and/or is an anti-inflammatory agent (see, e.g., U.S. Pat. Nos. 8,377,881, 8,357,402; 8,178,124; 8,143,218; etc.)

### 2. Biomedical Implants.

Biomedical implants of the present invention generally comprise a substrate and a coating on the substrate, which coating contains or carries nanoparticles as described herein. The coating may be porous or nonporous, flexible, rigid, or semirigid (e.g. a composite of a rigid material and a flexible material), and inert (stable) or biodegradable. In some embodiments the coating is porous and stable.

The nanoparticles may be positioned evenly throughout the coating, or may be distributed in the coating at varying densities, or located primarily on the external surface (or macro-surface) of the coating.

In some embodiments, such as a tissue expander or breast implant, the implant comprises an outer member forming an enclosure for a filler material. The outer member is typically a flexible inert, physiologically acceptable, polymer such as a silicone polymer. The filler material may be any suitable (preferably physiologically acceptable) liquid (including gel) material, such as saline solution, oils, polymers, etc. See, e.g., U.S. Pat. Nos. 8,382,833 and 5,964,803.

In some embodiments, the substrate comprises a hollow tube (e.g., a venous access tube, dialysis catheter, cerebrospinal fluid shunt).

In some embodiments, the substrate comprises a solid substrate (e.g. a tendon prosthesis or penile implant).

In some embodiments, the inert substrate comprises a stable or biodegradable material.

In some embodiments, the inert substrate comprises a flexible, natural or synthetic, organic polymer substrate.

In some embodiments, the inert substrate comprises a rigid metal (e.g., titanium), metal oxide (e.g., vanadium oxide), carbon fiber, ceramic (e.g., hydroxyapatite), natural or synthetic organic polymer, or composite thereof.

In some embodiments, the nanoparticles are electrically conductive.

In some embodiments, the nanoparticles contact one another in sufficient number to form a network or lattice.

In some embodiments, the coating comprises a stable or biodegradable, natural or synthetic, porous or nonporous, organic polymer having an average thickness of from about 50 nanometers to 5 millimeters.

While flexible polymeric substrates are generally preferred for carrying out the present invention, in some embodiments, particularly where rigid implants are susceptible to deleterious scar formation, the substrate may be a rigid material such as metal (e.g., titanium), ceramic, or a composite thereof. Examples of such substrates include intraocular stents, intravascular stents, etc.

In some embodiments the nanoparticles in or on the carrier or coating contact one another in sufficient number to form a lattice or network, which lattice or network may (or may not) define open spaces, pores, or interstices between the contacting nanoparticles.

The nanoparticles may be randomly distributed and/or oriented, or distributed and/or oriented in desired, nonrandom pattern.

Carrier polymers or coating polymers that can be used to carry out the present invention may be natural or synthetic, may be stable or bioabsorbable, and may be porous (or cell permeable) or non-porous. In general the carriers or coating polymers are preferably physiologically acceptable or biocompatible. Suitable examples include but are not limited to hyaluronic acid, alginate, collagen (including all types of collagen, including Type I, Type III, Type IV, and Type V), fibronectin, polylactide, polyethylene glycol, polycaprolactone, polycolide, polydioxanone, polyacrylates, polysulfones, peptide sequences, proteins and derivatives, oligopeptides, gelatin, elastin, fibrin, laminin, polymethacrylates, polyacetates, polyesters, polyamides, polycarbonates, polyanhydrides, polyamino acids carbohydrates, polysaccharides and modified polysaccharides, and derivatives and copolymers thereof (such as polylactide copolymers including PLGA) See, e.g., U.S. Pat. Nos. 6,991,652 and 6,969,480. Biological materials such as collagen, fibronectin, elastin, etc. may be from any suitable source, e.g., mammalian such as human, bovine, ovine, rabbit, etc.).

Nanoparticles (typically in a carrier or coating polymer) may be coated on the substrate by any suitable technique, including but not limited to those described in U.S. Pat. Nos. 8,227,076; 7,982,371; 7,666,494; etc.

The coating on the substrate may be any suitable thickness, but typically has an average thickness of from about 50, 100, 200 or 500 nanometers, up to 1, 2, or 5 millimeters.

As noted above, the present invention provides a method of inhibiting fibrosis, scarring, and/or fibrotic contracture of tissue contacting a biomedical implant (e.g., as described above) in a patient implanted with said biomedical implant, comprising: administering anisotropic nanoparticles to said tissue in an amount effective to inhibit fibrosis, scarring, and/or fibrotic contracture of said tissue. In some embodiments, the subject is administered antineoplastic chemotherapy or radiotherapy prior to or after receiving said biomedical implant. The administering step may be carried out by providing a coating on said implant, with said nanoparticles in or on said coating (e.g., wherein said coating comprises a stable or biodegradable, natural or synthetic, porous or nonporous, organic polymer having an average thickness of from about 50 nanometers to 5 millimeters).

### 3. Regenerative Templates.

In regenerative medicine, regenerative templates are known for a variety of different organs and tissues, including hollow and solid organs and tissues. All generally comprise a porous tissue scaffold into which cells from the subject may infiltrate, grow and/or proliferate. U.S. Pat. Nos. 8,226,715; 8,222,308; 8,167,955; 7,772,352; 7,731,756; 7,727,441;7,625,581; 7,622,299; 6,432,435; 5,842,477; etc. Scarring, fibrosis and/or contractures adjacent or around such templates after implantation in a subject can lead to a serious loss of tissue functionality.

Accordingly, and as noted above, the present invention provides a regenerative template for implantation in a subject, comprising: (a) a porous tissue scaffold; and (b) anisotropic nanoparticles on said scaffold in an amount effective to inhibit fibrosis, scarring, and/or fibrotic contracture of tissue contacting said template when implanted in a subject in need thereof.

In some embodiments, the scaffold is tubular or has at least one lumen, chamber or cavity formed therein (e.g., scaffolds such as for kidney, spleen, ducts such as bile ducts, ureters, and urethras, blood vessels such as arteries and veins, small intestine, central and peripheral nerve, etc., where an object is to minimize contracture both inside and outside the template). (Note that the lumen, chamber, cavity or orifice may be open or filled with a sacrificial material such as an erodible polymer for subsequent removal).

In some embodiments, the scaffold is unitary (e.g., a scaffold for a solid organ such as liver, or for a tissue such as muscle, skin, cartilage, etc., including composites thereof).

As previously, in some embodiments the nanoparticles are electrically conductive, and as previously in some embodiments the nanoparticles contact one another in sufficient number to form a network or lattice.

The nanoparticles may be coated on the surface of the scaffold by any suitable technique, such as by providing a coating on the scaffold (including both interior and exterior surfaces of the porous scaffold), with the nanoparticles in or on said coating. The coating may be a stable or biodegradable, natural or synthetic, porous or nonporous, organic polymer having an average thickness of from about 50, 100 or 200 nanometers to 1, 2, or 5 millimeters.

In other embodiments, the scaffold may comprise electrospun fibers (e.g., electrospun polymer fibers), with the nanoparticles coextruded with the fibers so as to protrude at least partially from the surface thereof, thereby coating the scaffold.

A method of inhibiting fibrosis, scarring, and/or fibrotic contracture of tissue contacting or infiltrating a regenerative template in a patient implanted with said regenerative template, can be carried out by administering anisotropic nanoparticles to said tissue in an amount effective to inhibit fibrosis, scarring, and/or fibrotic contracture of said tissue. Implantation of the template can be carried out in accordance with known techniques, depending upon the particular type of template being implanted. Administering of the nanoparticles can be carried out by including the nanoparticles in or on the scaffold prior to implantation, as indicated above, and/or by supplementing the scaffold by additional injection of nanoparticles, such as with a pharmaceutical composition containing the same as described below.

### 4. Treatment of Adhesions.

Adhesions are known to arise in a variety of different types of tissues, and from a variety of different types of injuries. See generally U.S. Pat. Nos. 7,144,588; 6,723,709 and 4,538,596. Hence, the present invention further provides a composition useful for inhibiting the formation of tissue adhesions in a subject in need thereof comprising, consisting essentially of, or consisting of: (a) a sterile pharmaceutically acceptable carrier; and (b) anisotropic nanoparticles in said carrier.

The carrier may be a fluid carrier (e.g., liquid, gel, gas, or combination thereof such as an aerosol or aerosolizable mixture). The nanoparticles may be included in the composition in any suitable amount: e.g., from 0.01, 0.1 or 1 percent by weight, up to 20, 40, 60 or 80 percent by weight, as desired, depending upon the particular type of tissue being treated and the specific carrier employed.

As previously, in some embodiments the nanoparticles are electrically conductive.

In some embodiments, the nanoparticles are included in said carrier in an amount sufficient to contact one another in sufficient number to form a network or lattice on a tissue susceptible to adhesions in a subject when applied thereto.

Where consisting essentially of the nanoparticles in the carrier, other typical minor ingredients for pharmaceutical compositions, such as preservatives, dispersants, stabilizers, propellants and the like, may also be included.

The compositions are useful in a method of inhibiting the formation of tissue adhesions in a subject in need thereof by topically administering the anisotropic nanoparticles to the tissue in an effective adhesion-inhibiting amount.

In some embodiments, the tissue is endogenous or native tissue in said subject and which tissue is afflicted with an injury (e.g., mechanical trauma, burns, ischemia, radiation injury, incisions, lacerations, crushing injury, surgical incision or intervention).

In some embodiments, the tissue is native intra-thoracic, intra-abdominal, or intra-cranial tissue (including but not limited to plural and epicardial membranes, lung, heart, diaphragm, intestinal, spleen, stomach, omentum, brain, dura mater, bone, etc.)

The administering step may be carried out by any suitable technique, but is preferably carried out by contacting a pharmaceutically acceptable fluid carrier (e.g., liquid, gel, gas, or combination thereof such as an aerosol) to the tissue, said carrier having said nanoparticles therein. In some embodiments, the nanoparticles are electrically conductive nanoparticles, and in some embodiments the nanoparticles are applied to the tissue in an amount sufficient to contact one another in sufficient number to form a network or lattice thereon.

### 5. Inclusion of Adhesion Inhibitors.

In biomedical implants as described above, in regenerative scaffolds as described above, in compositions for treatment of adhesions as described above, and in methods of treatment or use as described in connection with all of the above, cell adhesion-inhibiting compounds may also be included, or concurrently administered, in an effective-adhesion inhibiting amount.

Numerous cell adhesion inhibiting compounds are known, examples of which include but are not limited to hyaluronic acid, Arg-Gly-Asp (RGD) peptides, anti-RGD binding antibodies (T. Vassilev et al., Blood 93, 3624-3631 (1999); Peribysin J and macrosphelide M (T. Yamada, J. Antibiot. 60(6): 370-375 (2007); etc. Additional examples of cell adhesion inhibiting compounds include but are not limited to those set forth in U.S. Pat. Nos. 5,629,294 and 6,129,956, and in US Patent Application Publication Nos. 20110182989, 201200888832, and 20130252921, the disclosures of which are incorporated by reference herein in their entirety.

Adhesion inhibitors may be coupled to carriers, coatings, or scaffold materials in accordance with known techniques; simply mixed with coatings or carrier; or administered separately, but concurrently, to the same tissues (e.g., by injection, spraying of exposed tissue, etc.) In some embodiments, the adhesion inhibitors are coupled (covalently or noncovalently, and directly or through an intervening linking group) to the nanoparticles. Such coupling may be carried out in accordance with known techniques. For example, adhesion inhibitors that contain a carboxylic acid group may be coupled to carboxylated nanotubes through a reduction reaction).

The present invention is explained in greater detail in the following non-limiting Examples.

### EXPERIMENTAL

Collagen gels provide a standard mechanism to evaluate tissue remodeling and have been widely used to reproduce the activity of mesenchymal cells and fibroblasts in vitro.^{[6, 20-23]} The cells are known to traverse and interact with collagen fibers and we hypothesized that we could alter cellular contractile activity by doping the gels with fiber-like materials such as carbon nanotubes. MWNT are essentially larger versions of SWNT; MWNT are thicker than SWNT because they have multiple layers of carbon atoms, and this added thickness allows for stabile nanotubes at longer lengths. Both types of nanotubes have a very high aspect ratio—the measurement of their length to their width—ranging from roughly 100 to 2000, which causes these materials to act as fibers. In addition, carbon nanotubes have previously been used to alter the behavior of osteoblasts, which also interface closely with collagen, to promote proper bone healing.^{[24]} To investigate the effect of nanoparticle shape on fibroblast activity, we used spherical ultrafine carbon black (17 nm diameter) as a control along with single- and multi-wall nanotubes (SWNT and MWNT, respectively). The SWNT used in this study were 100-1000 nm in length and 0.8-1.2 nm in diameter, while the MWNT were 3-30 μm in length with a diameter of 13-18 nm (data provided by the manufacturers).

Materials and Methods

Cell Culture.

The cell line human embryonic palatal mesenchyme (HEPM) was purchased (ATCC, Manassas, Va.) and cultured according to the vendor's specifications in DME/High Modified media (HyClone, Logan, Utah) with 10% fetal bovine serum, 1% L-glutamine, and 1% penicillin and streptomycin (each from Life Technologies, Carlsbad, Calif.). Cells were used between passages 15 and 20. Viable cells were counted using a hemocytometer and trypan blue exclusion.

Nanoparticle Solutions.

The effect of nanoparticle shape on fibroblast contraction was evaluated using 17 nm spherical carbon black (Cabot, Boston, Mass.), SWNT (Unidym, Sunnyvale, Calif.) created from high pressure carbon monoxide conversion synthesis (HiPCO), and MWNT (Cheap Tubes Inc., Brattleboro, Vt.). None of these particles were oxidized or otherwise functionalized in any way. Each of the three types of nanoparticles was tested at three concentrations: 100 μg/mL, 10 μg/mL, and 1 μg/mL. Because the nanoparticle solution comprised 10% of the total gel volume, these concentrations are equivalent to 1%, 0.1%, and 0.01% nanoparticle content respectively by weight per volume (w/v).

The nanoparticles were sterilized by adding them to phosphate buffered saline (PBS) (Sigma, St. Louis, Mo.) and then centrifuging at 14,000 rpm for 30 minutes in an Eppendorf 5418 centrifuge (Eppendorf, Hamburg, Germany). The supernatant was then removed and the particles were resuspended in 70% ethanol in water before being spun again at the same speed and duration. The supernatant was again removed after this process and the nanoparticles were resuspended in 1×PBS and 1 mg/mL Pluronic F-127 (Sigma, St. Louis, Mo.) at a concentration of 100 μg/mL. Nanoparticle solutions were then horn sonicated using a Branson digital sonifier with ⅛″ tapered microtip attachment (Branson Ultrasonics Corporation, Danbury, Conn.) for 5 minutes at 20% amplitude with a pulse duration of 2 seconds and a 50% duty cycle. The horn sonicator tip was sterilized with 70% ethanol in water before and after each use. A Beckman Coulter DU 730 Life Science UV/Vis spectrophotometer (Beckman Coulter, Inc., Brea, Calif.) was used to evaluate the optical density of the nanoparticle solutions at 808 nm in order to confirm nanoparticle concentration by comparing a 1:10 dilution of the solution to a known 10 μg/mL solution of the non-sterilized and well suspended nanotubes. The sterile solution was then serially diluted to generate the lower two concentrations. Solutions were stored in a 4° C. refrigerator and sonicated again for 1 minute of the same cycle immediately before they were used in the gels.

Gel Preparation.

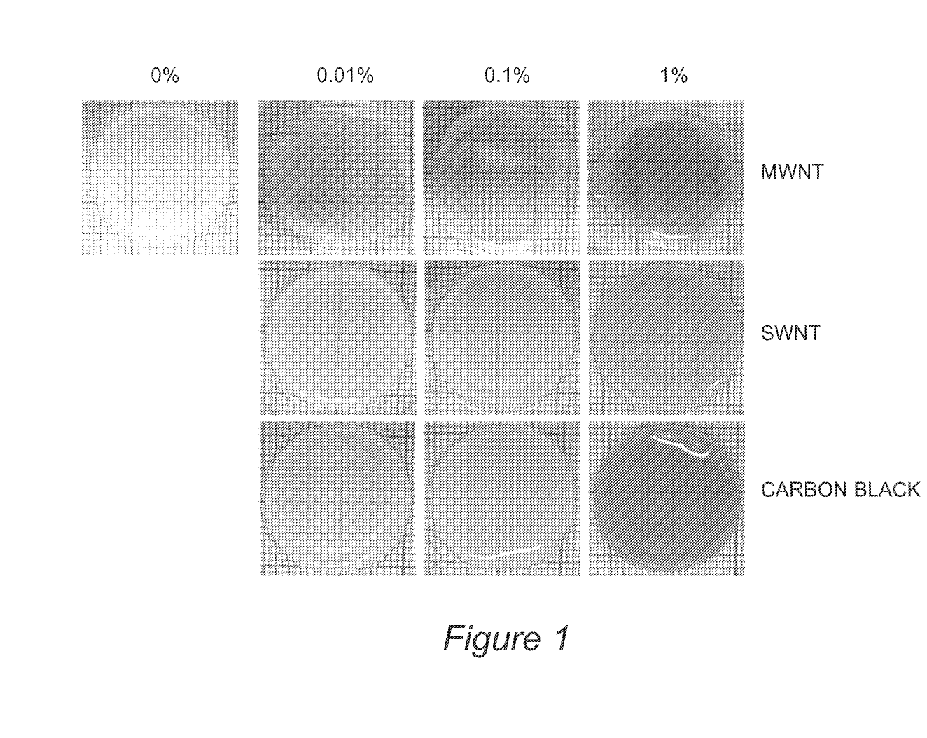

Gels were formed in 12-well plates by combining the following solutions in each well in the order they are listed: 100 μL 10×PBS, 20 μL 1M sodium hydroxide, 130 μL cold nanoparticle solution, 800 μL cold 5 mg/mL rat tail collagen type I diluted in 0.02M acetic acid from high concentration collagen (BD Biosciences, San Jose, Calif.), and 250 μL cell solution of 4×10⁶ cells/mL in media. All gel types were made in triplicate, with three gels of each concentration for each of the three nanoparticle types. Positive control gels were made without nanoparticles in the PBS/Pluronic solution and negative control gels were made without cells, using only media for the cell solution. These solutions were then gently pipetted to mix and bubbles were aspirated from the gels. During development of the protocol, it was discovered that immediate incubation caused aggregation of the nanoparticles. Brightfield microscopy was used to document this phenomenon. As a result, the gels described here were allowed to initially set in a hood at room temperature for half an hour and were then transferred to a 37° C. incubator for another half an hour. This method resulted in no readily apparent nanoparticle aggregation, but the presence of smaller aggregates was later investigated with polarized light microscopy using an Axioplan 2 microscope (Zeiss, Oberkochen, Germany). After an hour, the gels had fully set and were gently removed from their wells individually to be photographed in a 38 mm plastic culture dish against millimeter-square graph paper. The gels were then replaced in their original wells and topped with 2 mL media per gel. Photographs were taken of each gel every day for one week after gel formation. The media was changed every other day starting the first day after gel formation. Representative gels can be seen in FIG. 1.

Contraction Analysis.

The area of each gel on each day was calculated based on two perpendicular measurements of the diameter of the gel. Contraction was assessed by dividing the area of cellularized gels by their non-cellular negative controls. A one-way ANOVA was then applied to this normalized contraction data using SigmaStat version 3.5. Post-hoc testing was also performed in SigmaStat using the Holm-Sidak method with each value being compared to the cellularized gel without nanoparticles, i.e. the positive control. A two-way ANOVA was also performed on the raw, non-normalized data to determine the relative contributions of the cells, nanoparticles, and the interaction of the two to the observed effect on contraction.

Cell Viability.

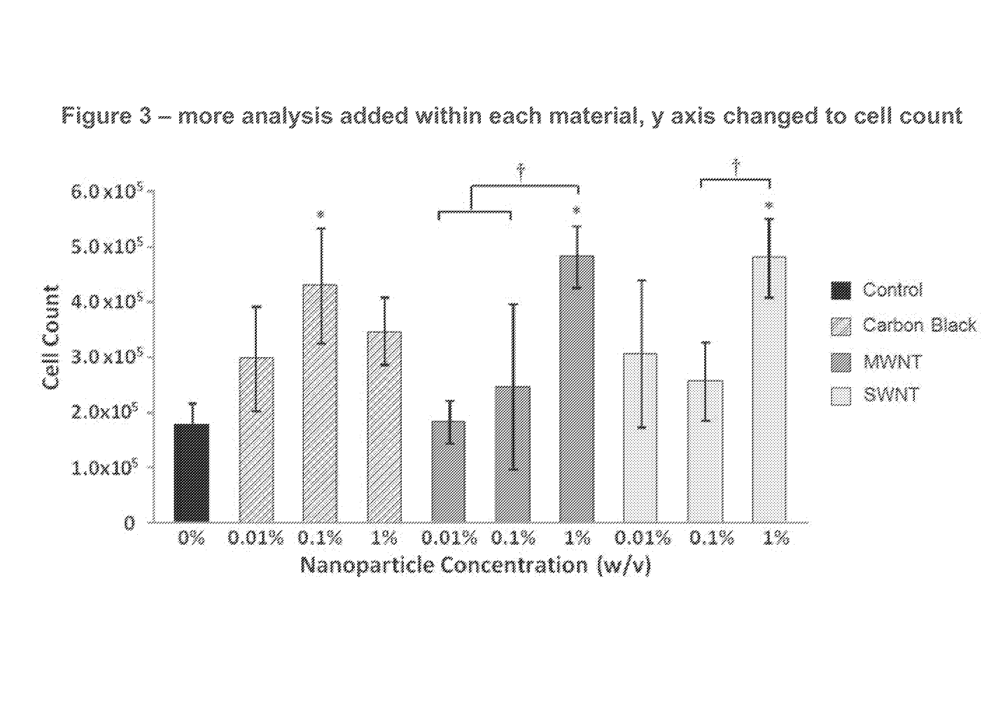

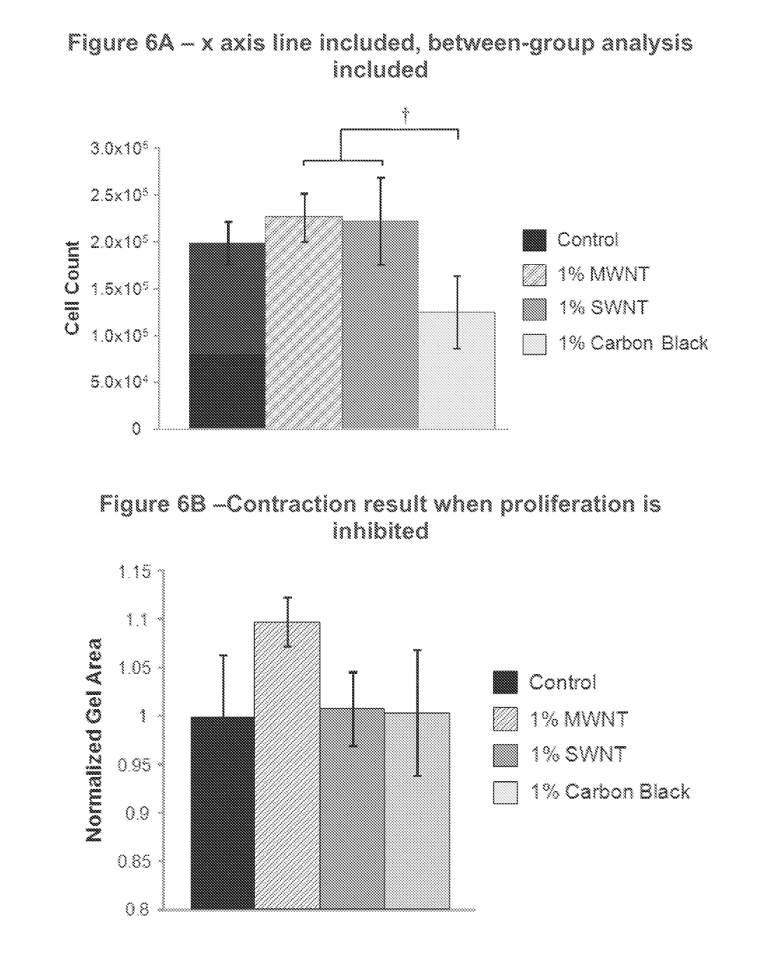

The gels were evaluated after the one week incubation period had concluded. The gels were incubated in 2 mg/mL collagenase IA (Sigma, St. Louis, Mo.) in media for 90 minutes until they were completely dissolved. The solution was homogenized with vigorous pipetting and then an aliquot of this solution was counted using a hemocytometer and trypan blue exclusion to measure cell viability. The volume of solution in each well was also measured so as to generate a full cell count per gel. Average cell counts from the collagenase digestion of each gel type were each normalized by the average cell count from the positive control gels. This method was used to generate the data in FIGS. 3 and 6. A one-way ANOVA was then applied to the overall viability data set. Post-hoc testing was also performed using the Holm-Sidak method.

Other gels were made for the purposes of staining; these gels were not dissolved but instead underwent a Live/Dead assay (Life Technologies, Carlsbad, Calif.). Based on a similar protocol,^{[21]} the gels were washed with 2 mL 1×PBS per gel and incubated for 40 minutes to remove media, then the PBS was replaced with 1 mL of a solution of 5 μM ethidium homodimer and 5 μM calcein in PBS, concentrations that were successful in another protocol.^{[33]} The gels were incubated for 45 minutes before this solution was removed and 2 mL fresh PBS was added per gel. The gels incubated for a final 20 minutes before they were transferred to chamber slips for confocal imaging. Argon and Helium-Neon lasers were used to excite the calcein dye at 488 nm and the ethidium homodimer dye at 543 nm. The fluorescence was imaged at 505-530 nm for the calcein and >560 nm for the ethidium homodimer. Each gel was viewed under 10× magnification using a Zeiss Axiovert 100 M confocal microscope with an attached laser scanning microscopy unit, LSM 510 (Zeiss, Oberkochen, Germany). The number of cells shown in each confocal image was later counted to facilitate quantitative comparison of cell count trends.

Proliferation.

To investigate the proliferative behavior of the cells encapsulated in these gels, mitosis was blocked with the antiproliferative chemical cytosine β-D-arabinofuranoside (also known as Ara-C; Sigma, St. Louis, Mo.). Based on a previous protocol,^{[34]}10 μL of a 200 μg/mL solution in sterile water was added to each gel solution before mixing. The gels were then maintained identically to the other gels and evaluated using the trypan blue cell counting protocol and contraction analysis described in the previous two sections.

Actin Staining.

Fibroblast phenotype was investigated by measuring the content of the three different actin isoforms in the cytoskeleton of the cells in each gel type. Each gel type was tested in triplicate at the end of 7 days of incubation to allow for any cytoskeletal changes to fully develop. The media was removed and replaced with PBS for 10 minutes to wash away any remaining media. The PBS was then removed and gels were fixed with 4% paraformaldehyde in PBS for 30 minutes, washed with more PBS, and then permeabilized with 0.2% Triton-X-100 (Fisher Scientific, Waltham, Mass.) for 15 minutes. After another PBS wash, the gels were blocked with 2.5% BSA for 90 minutes. Five millimeter diameter samples were taken from each gel in an area directly between the center and the edge of each gel using a round biopsy punch; samples were transferred to a 96-well plate after collection. Fluorescently tagged antibodies, each at a 1:40 dilution applied simultaneously for 1 hour, were applied to the samples after another PBS wash. The antibodies were anti-β actin fluorescein isothiocyanate (FITC) (Abcam, Cambridge, England), rhodamine phalloidin (Life Technologies, Carlsbad, Calif.), and anti-ACTA2 conjugated to HiLyte plus 647 (LifeSpan Biosciences, Inc., Seattle, Wash.). These correspond to β actin, F actin, and α actin respectively. The ACTA2 isoform is specific to α smooth muscle actin, which is used to specifically determine the presence of myofibroblasts and does not bind to α skeletal muscle, α cardiac muscle, or α or γ cytoplasmic actin. After a final PBS wash, the fluorescence was then excited/measured at 488 nm/525 nm, 543 nm/570 nm, and 633 nm/670 nm using an Infinite M200 plate reader (Tecan, Männedorf, Switzerland) to generate quantitative data. The gels were also viewed at 40× magnification using the same confocal microscope as was used for the Live/Dead assay. On the confocal microscope, the stained samples were excited at 488 nm, 543 nm, and 633 nm and read at 505-530 nm, 565-615 nm, and >650 nm.

Reactive Oxygen Species Assay.

A hypoxanthine/xanthine oxidase assay was used to gauge any antioxidant effect of the nanoparticles. The following stock solutions were formed: 1.95 mM cytochrome C in PBS, 1 mM hypoxanthine in 0.9 mM NaOH in PBS, 10200 U/mL superoxide dismutase in PBS, 6000 U/mL catalase in PBS, and 167.5 mU/mL xanthine oxidase in PBS. These stock solutions were then combined to form experimental solutions A, B, and C. Solution A included 320 μL of catalase, 320 μL hypoxanthine, 755 μL cytochrome C, and 14.6 mL PBS. Solution B was 57 μL xanthine oxidase and 743 μL PBS. Solution C combined 57 μL xanthine oxidase, 90 μL superoxide dismutase, and 653 μL PBS. The experimental solutions were then mixed to yield reactive oxygen species. Each gel was washed with PBS and a baseline absorbance measurement was taken at 550 nm using the same plate reader as was used in the actin assay. Each gel then received 200 μL of solution A and an equal volume of either solution B or C. Five triplicate sets of gels were tested after they were incubated for 7 days; one set of gels for each nanoparticle type and one set of positive control gels (i.e. those with cells but without nanoparticles) were activated with solution B. Another set of otherwise positive control gels was tested with solution C to act as the negative control for this experiment, since superoxide dismutase (SOD) quenches the reactive oxygen species. The absorbance was then measured every 5 minutes for 90 minutes total to gauge the long-term effects of the nanoparticles on the cells. Each data point was evaluated as the change from the baseline reading. The plate reader was maintained at 37° C. and the plate was not disturbed during this time.

Compression Testing.

To determine the Young's modulus of the nanotube-doped collagen gel, acellular gels were made similarly to the description in the “gel preparation” section. Another control gel with a higher concentration of collagen was also used to better gauge the effect of stiffness changes resulting from changing the collagen alone. For this set of gels, an 8.93 mg/mL stock collagen solution was used in gel formation instead of the normal 5 mg/mL stock. All of the gels were made with half the normal volume of solution used to make the gels and in 48-well plates instead of 12-well plates, resulting in gels that were chemically identical to the other gels tested but that were taller and narrower than the original wide, flat gels. These dimensions facilitated compression testing, which is more accurate if no one dimension is much larger than another. Also, more data can be gleaned from a thicker sample because it can be compressed more thoroughly to evaluate higher strains. The height and diameter of each gel was measured with Vernier calipers prior to each test. Each concentration of each material type was evaluated in triplicate.

Samples were tested using an ElectroForce mechanical tester with compression platens, a 1 kg load cell, and WinTest 4.1 software (all from Bose, Eden Prairie, Minn.). Samples were tested at 0.01 mm/s for 1.5 mm and data was collected at 20 points per second. The data was analyzed by converting the load and displacement data into stress and strain values using the individual dimensions for each gel. Stress vs. strain plots were then generated in Excel (Microsoft, Redmond, Wash.). Strains of 10% or less were evaluated to only consider the linear region of the stress-strain curves, which indicate the region of elastic deformation applicable to Young's modulus calculations. Linear trendlines of these plots were fit using Excel and the slope was taken as Young's modulus, also called the elastic modulus. Most of the trendlines had correlation coefficients (R²) of 0.97 or better, but none were below 0.86 (out of a possible 1.0). One- and two-way ANOVAs were used to analyze the results.

Results.

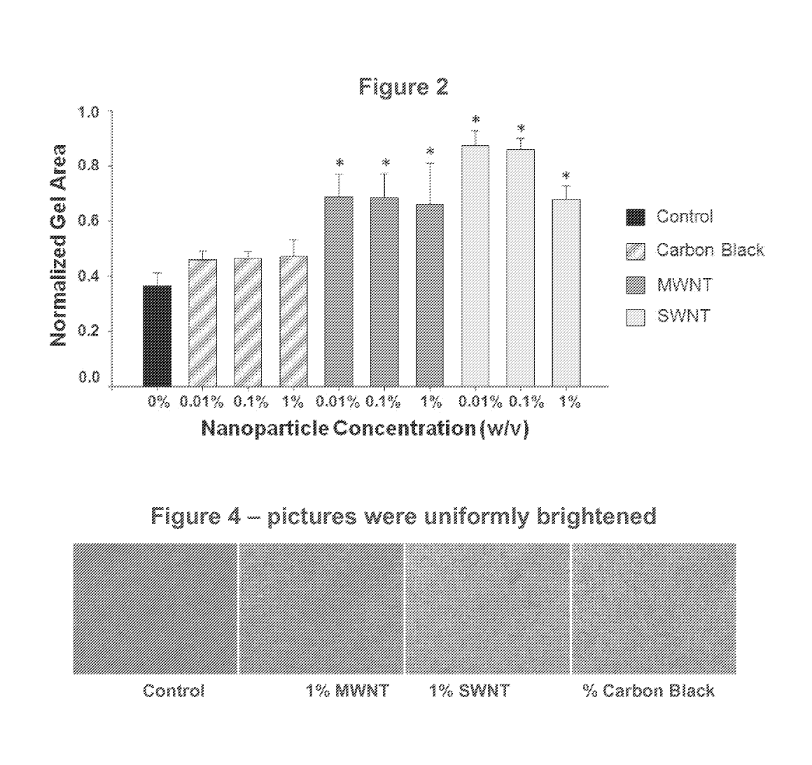

Gels darkened with increasing concentrations of incorporated nanoparticles as shown in FIG. 1. At the end of one week, contraction was observed in every gel regardless of nanoparticle concentration, but only the MWNT and SWNT gels were statistically different in area from the control gels as shown in FIG. 2. While ultrafine carbon black, as was used here, has been reported to inhibit contraction via adsorption of pro-contractile fibronectin and TGF-β,^{[6]} the Pluronic F-127 coating prevented the carbon surfaces from adsorbing any other compounds. Within gel types, no concentration of carbon black contracted more significantly than any other, nor did any concentration of MWNT. The highest concentration of SWNT contracted more significantly than the lower two concentrations on a p<0.05 level. Between gel types, the highest concentrations of MWNT and SWNT contracted to a statistically similar extent, while the lower two concentrations of SWNT contracted significantly less than the same concentrations of MWNT (p<0.05). Because contraction is a cell-mediated process, one might expect to see a decrease in contraction if the nanoparticles, and particularly the nanotubes, were cytotoxic since dead cells cannot contract. Interestingly, as shown in FIG. 3, all of the nanoparticle gels resulted in an increased number of viable cells compared to the non-nanoparticle control gels. The highest concentrations of MWNT and SWNT had nearly 3 times as many cells as the control.

MWNT and SWNT increase cell viability only at the highest concentration tested, while carbon black increases cell viability only at the intermediate concentration. While the 0.1% carbon black gel does have a statistically significant increase in the number of viable cells over the control gel, the 0.1% carbon black gel is not significantly different from either of the other two concentrations of carbon black gels, neither of which are different from the control. In the MWNT group, the 1% gel is significantly different from each of the lower concentrations, which are not significantly different from one another. The SWNT group is somewhat less clear-cut, with the 0.1% and 1% concentrations being significantly different from one another, but the 0.01% group not being different from either of the other two concentrations. These data show that the effect of the nanotubes on contraction changes with dose.

We investigated the role of the nanoparticles in the interaction using a two-way ANOVA, which assesses the contribution of each factor to the overall variance in the data. It was found that gel contraction is affected by the inclusion of cells in the gel, which would be expected from a cell-mediated process. The relationship between the inclusion of cells and gel contraction was highly significant; data is available in table 1.

TABLE 1
The effects of the cells, nanoparticles, and the interaction of
the two each contribute significantly to the observed contraction.
Significance levels for each permutation of the two-way ANOVA output.
Significant values on a p < 0.05 level are in italics.

| | Cells Alone | Nanoparticles Alone | Interaction |
|---|---|---|---|
| Carbon Black | p < 0.001 | p = 0.058 | p = 0.022 |
| MWNT | p < 0.001 | p = 0.008 | p = 0.003 |
| SWNT | p < 0.001 | p < 0.001 | p < 0.001 |

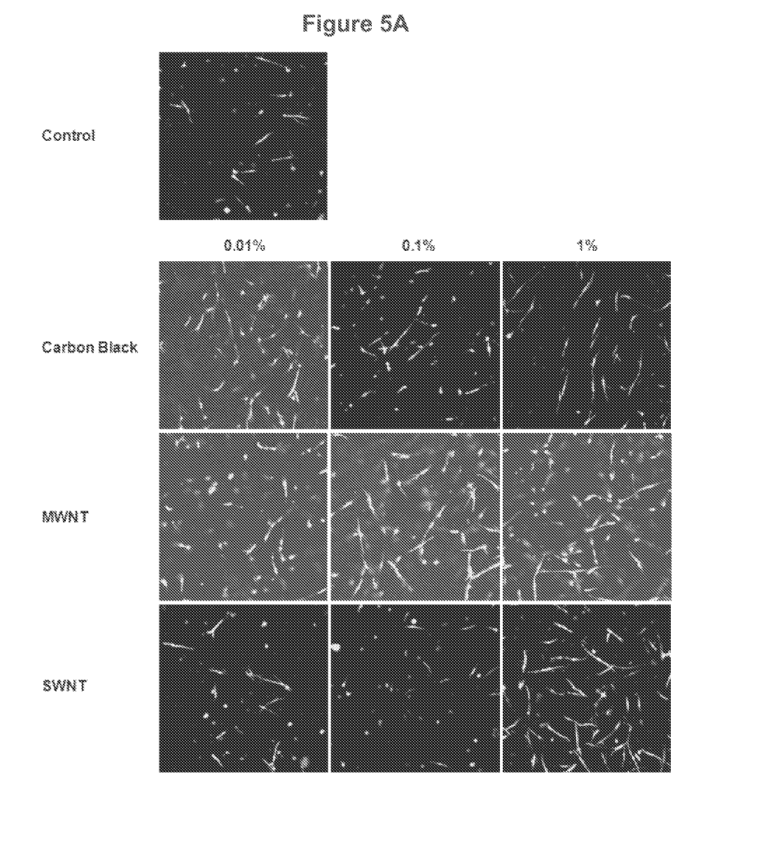

The effect of the nanoparticles alone was found to be significant for the MWNT and SWNT, but not for the carbon black. The interaction of the nanoparticle and cell effects was also found to be significant for each material, which further supports our theory that the nanoparticles are interacting with the cells and mediating their contraction on a cellular level. To further buttress this result, the gels were viewed with polarized light microscopy at day 7 to test for the presence of nanoparticle aggregates. As shown in FIG. 4A, no nanoparticle aggregates were detected even at the end of compaction. Because the nanoparticles are well dispersed to at least this extent, they should be well incorporated into the matrix and therefore better able to interact with cells on an individual level.

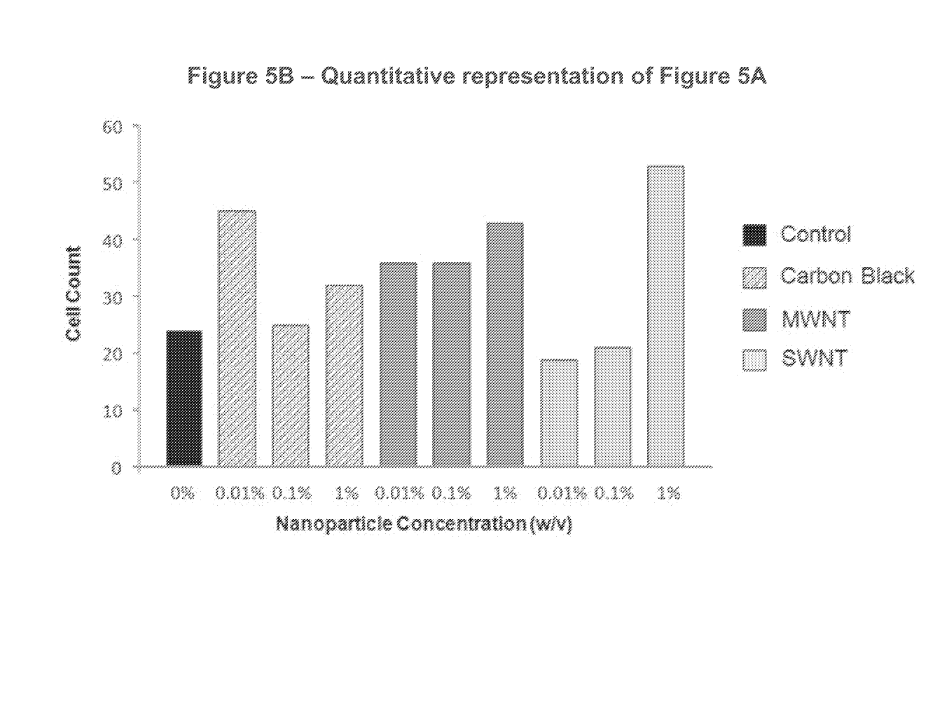

To investigate the cause of the differences in cell number at day 7, as shown in FIG. 3, viability was also investigated at day 3 to evaluate if the differences were due to a large set of apoptosing cells at the end of the incubation period, as might be expected from myofibroblasts that have successfully contracted a wound. All gels contained numerous healthy cells after 3 days of incubation, but some materials had many more cells than others, as shown in FIG. 5. Counts of the cells in each individual image from FIG. 5A are plotted more quantitatively in FIG. 5B. The confocal images from day 3 reflect the trends seen in the day 7 cell counts that nanoparticles increase cell viability and the highest concentrations of nanotubes have the strongest effect. Because the trends are the same for days 3 and 7, the results must be due to an effect that is consistent over the course of the experiment and cannot be attributed to increased cell death late in the experiment. To determine the mechanism of this consistently increased viability, proliferation was blocked when a new batch of gels were made. As shown in FIG. 6A, the increase in the number of viable cells is removed when proliferation is blocked, proving that the difference in viability is due to increased proliferation. Interestingly, it was also noticed that this inhibition removes the differences in contraction between the different gel types (FIG. 6B).

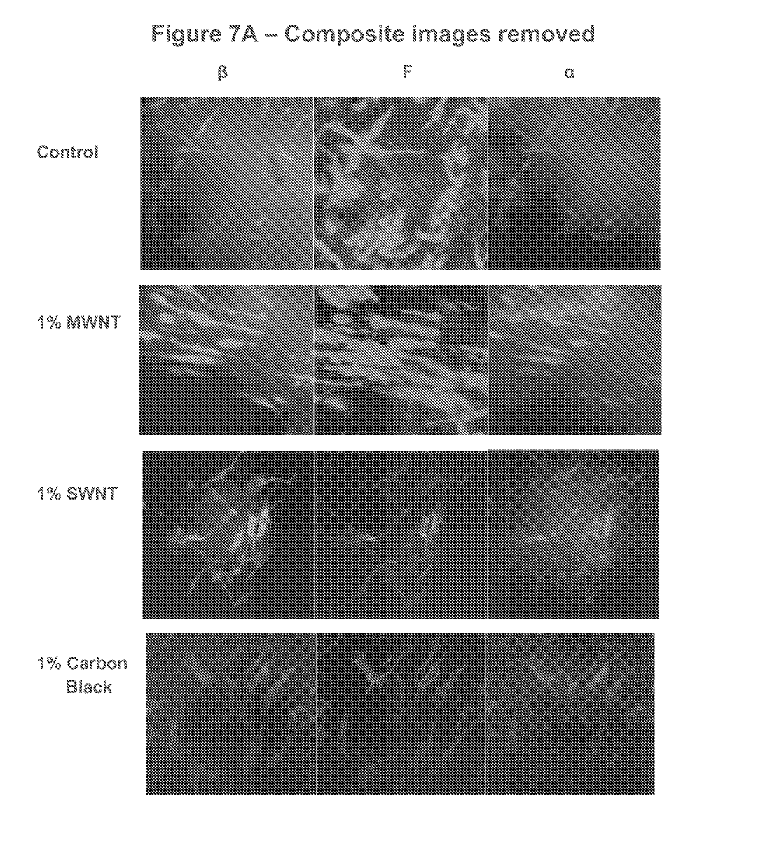

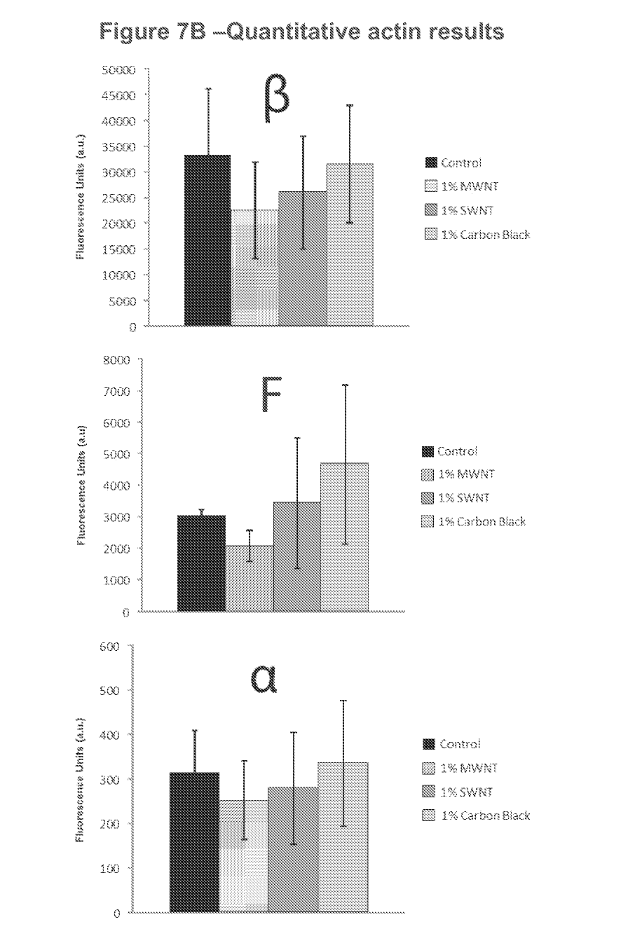

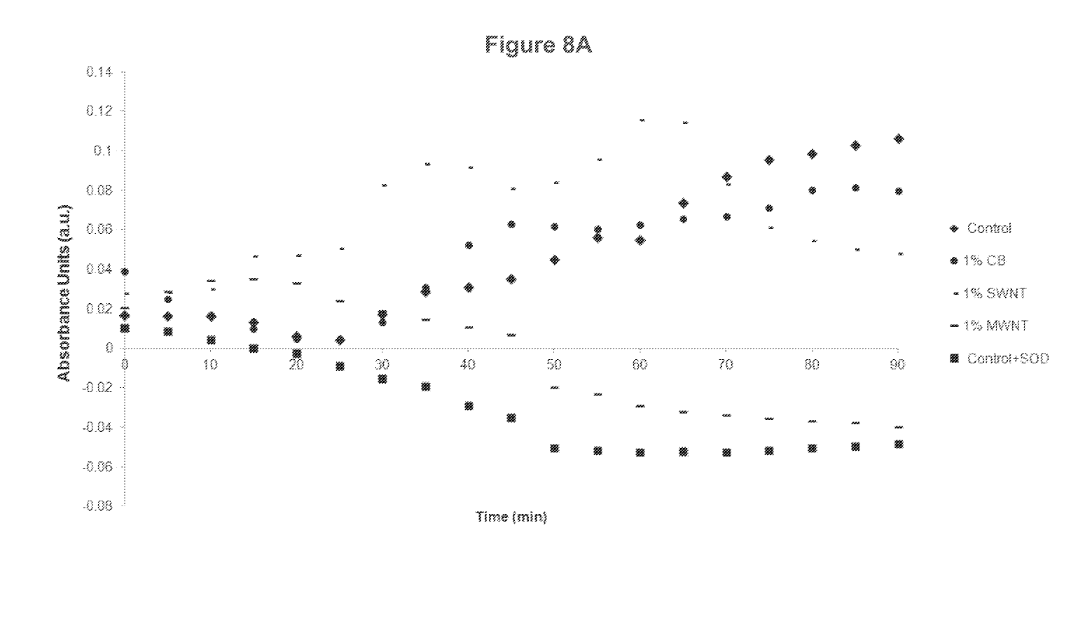

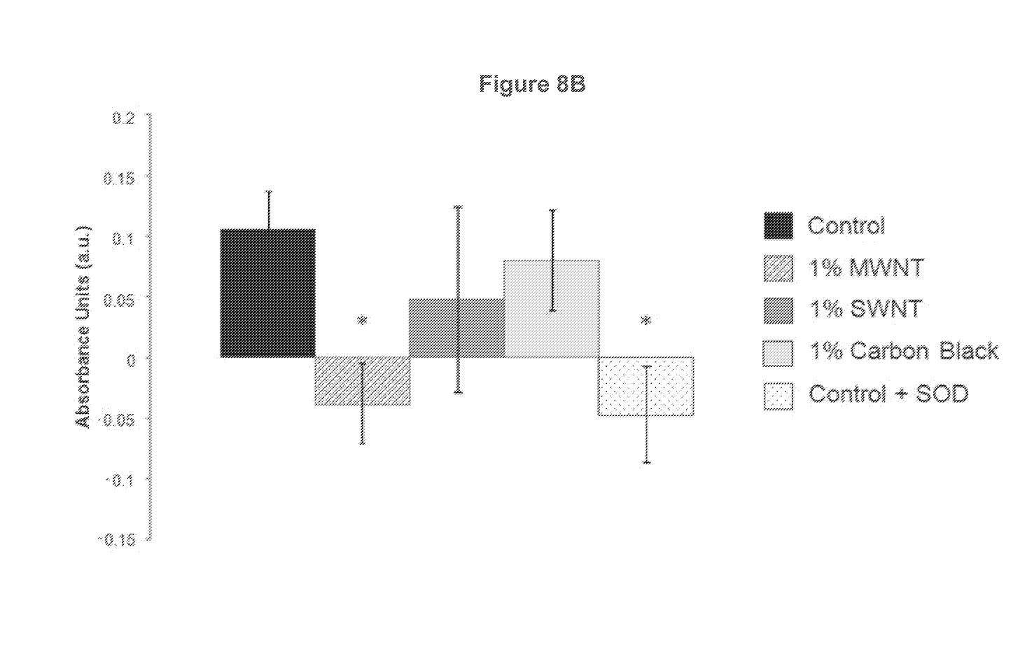

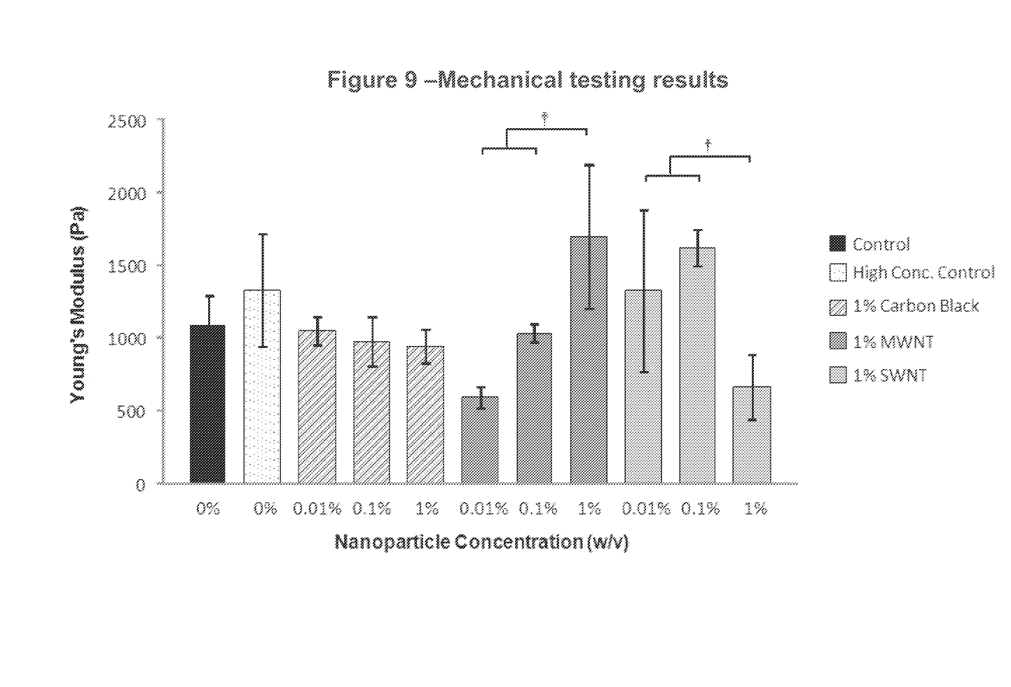

The mechanism of the differential cell responses to each gel type was also investigated. It was evaluated initially with an actin assay which showed that each gel type expressed β, F, and α actin. (FIG. 7) There was no significant difference in actin isoform expression between materials. These data are represented in FIGS. 7A and 7B respectively. While the β and F actin is expected to be the same for all groups, the α smooth muscle actin result was less expected, since only myofibroblasts express α actin and generally myofibroblasts increase contraction. Many pathways mediate the myofibroblastic phenotype, however, and reactive oxygen species have been correlated with the presence of this phenotype through both TGF-β and integrin signaling pathways.^{[36-37]} To further probe the mechanism of the nanotubes' effect, a reactive oxygen species assay was performed. (FIG. 8) The relatively long time scale of the assay is justified because it was investigating the longer-term effects of reactive oxygen species on the cells and, as shown in FIG. 8A, the assay does take a while to stabilize in the 2-3 mm thick collagen gels. This is likely because the hypoxanthine/xanthine oxidase assay generates extracellular free radicals but the radicals are intracellularly quenched.^{[38]} As shown in FIG. 8B, the MWNT exhibit an effect similar to the antioxidant superoxide dismutase, whereas the carbon black nanoparticles have no antioxidant effect. The SWNT had too large of a standard deviation to ascertain to which group it belongs. The mechanical properties of each gel were also evaluated as a possible mechanism of action. (FIG. 9) None of the gels were found to be different than the control in a one-way ANOVA. However, a two-way ANOVA showed a now familiar trend: the only significant changes were for the highest concentrations of nanotubes. The carbon black gels showed no difference from one another at any concentration. The 1% MWNT gels had a significantly higher Young's modulus than the lower two concentrations of MWNT gels. The 1% SWNT gels were also significantly different from the lower two concentrations of SWNT gels, but the 1% gels were significantly less stiff.

Discussion.

It has previously been demonstrated that carbon nanoparticles non-specifically interact with collagen through hydrophobic-hydrophobic interactions.^{[39]} Theoretically, perfectly dispersed nanoparticles should not change the optical density of the gel, and a darkening of the gel suggests aggregation of the nanoparticles.^{[40]} The gels generally started out as almost clear, though the carbon black gels were somewhat darker. We observed the carbon black gels become noticeably darker during their significant compaction and we also observed this effect to a lesser degree with the nanotube gels. However, the gels still showed no aggregation when tested at the end of their incubation period, which as a result of the compaction process is the point when they are most likely to contain aggregates (FIG. 4).

The data show that the embedded nanoparticles change both the matrix of the gel itself and the cells surrounding the nanoparticles. The results of the two-way ANOVA show that the effect of the nanotubes alone significantly affects the contraction of the gel, which would be the case if the nanotubes were somehow affecting the collagen matrix and making it more difficult or less favorable for the cells to contract the gel. Because the nanoparticles are well dispersed instead of aggregated, they are better able to interact with cells on the nanoscale and also interact with all the cells in a more uniform fashion.

The MWNT act as antioxidants, and similar results have been reported for other PEGylated carbon nanoparticles, such as hydrophilic carbon clusters (PEG-HCCs).^{[30]} Studies with other antioxidants have shown that a decrease in reactive oxygen species causes an overall decrease in inflammatory processes, which results in a decrease in collagen gel contraction.^{[37]} While most of the ROS data can be explained, the SWNT data is more mysterious. Based on the results, the SWNT data was repeated later with a fresh batch of gels, but the same value and standard deviation were observed (data not shown). Other authors have shown that decreased gel contraction is related to a decrease in ROS with a subsequent decrease in a smooth muscle actin expression,^{[37]} which makes our finding of unchanged α smooth muscle actin expression with decreased contraction even more intriguing.

The elastic modulus of the cell substrate has also been shown to greatly affect the behavior of cells.^{[41]} The addition of carbon nanoparticles to a material creates a composite with an increased modulus, but the change in modulus is usually observed at significantly higher nanoparticle fractions than were tested here.^{[42-44]} In addition, it is very difficult to mechanically test hydrated gels, as the extrusion of the fluid from the gel has its own contribution that can blunt the detection of actual differences. However, there are other ways for the particles to affect gel properties. High aspect ratio particles such as MWNT and SWNT require a smaller number of particles than low aspect ratio particles such as carbon black in order for a network of the nanoparticles to be present throughout the matrix. This lattice network of interacting nanoparticles is needed to significantly change the properties of the material; until that point, only much smaller differences will be seen. The concentration of nanoparticles or other dopants that results in the formation of such a network is known as the percolation threshold. The MWNT and SWNT should experience this transition near 0.5 wt %, but the carbon black would require closer to 20 wt %.^{[35, 45]} We believe that the differences we observe in this work are attributable to crossing that percolation threshold in the 1% MWNT and 1% SWNT gels.

A lattice increases conductivity when there are enough conductive elements that touch each other to create unbroken electrical paths across or throughout a substrate. Fibroblasts have been shown to increase cell adhesion, spreading, and proliferation on conductive substrates,^{[46]} and a random matrix formed by nanotubes corroborates evidence from other authors that Pluronic F-127 wrapped carbon nanotube-doped gels enhance the electrical conductivity, leading to improved cytocompatibility.^{[26]} In the formation of a lattice, there is little difference in the observed effects below a critical threshold of conductor connectivity, but a significant step occurs once the threshold is crossed. The nanotube gels show a greatly increased viability at the 1% concentration after flat or minimal cell count growth at the lower concentrations. While the 0.1% carbon black gels seem to significantly increase cell counts in FIG. 3, this trend is actually reversed in FIG. 5 with the 0.1% carbon black gels exhibiting the lowest cell counts of that gel type. As a result, we believe that carbon black shows a concentration-independent increase in viability. Because cell viability varies strongly with substrate conductivity, the dose dependence of the nanotubes on cell viability demonstrates this percolation threshold is reached at the highest nanotube concentration tested (1%) and is lacking in the spherical carbon black gels. This conclusion is strengthened by the results of the proliferation study, which show that the difference in viability between the various nanoparticle dopants is due to proliferation.

By incorporating a nanotube lattice into the gels, cells have a more difficult time contracting the gels, but also have more scaffold-like substrate on which to proliferate. This combination of decreased contraction but increased proliferation would be well suited to aid in healing of wounds that have reached the connective tissue, such as deep burns. Such an application should speed healing time and decrease the likelihood of a scar contracture developing from the injury.

### CONCLUSION

Carbon nanotubes are potent inhibitors of mesenchymal cell-mediated contraction even though the nanotubes significantly increase the number of viable cells available to contract. A significant mediator of this effect is the aspect ratio of the carbon nanoparticles. The fiber-like shape of the nanotubes leads to important consequences in their biological interactions, allowing them to generate a matrix within the collagen gel at the concentrations tested. The antioxidant ability of the MWNT also contributes to the effect and would serve to further decrease inflammation in in vivo applications. Because fibrosis yields a number of detrimental effects by creating a disorganized ECM and altering cell-ECM interactions, the ability of carbon nanotubes to restructure the matrix and scavenge reactive oxygen species offers a new tool to treat a variety of fibrotic diseases from scar contractures to cancer metastases.

### REFERENCES

[1] Nielsen M S. Myocyte-fibroblast interactions—risky connections. Heart Rhythm. 2009, 6, 1650-1.
[2] Yang L, Hashimoto K, Tohyama M, Okazaki H, Dai X, Hanakawa Y, et al. Interactions between myofibroblast differentiation and epidermogenesis in constructing human living skin equivalents. J Dermatol Sci. 2012, 65, 50-7.
[3] Adamson I Y, Hedgecock C, Bowden D H. Epithelial cell-fibroblast interactions in lung injury and repair. Am J Pathol. 1990, 137, 385-92.
[4] Desmouliere A, Redard M, Darby I, Gabbiani G. Apoptosis mediates the decrease in cellularity during the transition between granulation tissue and scar. Am J Pathol. 1995, 146, 56-66.
[5] Goel A, Shrivastava P. Post-burn scars and scar contractures. Indian J Plast Surg. 2010, 43, S63-71.
[6] Kim H, Liu X, Kobayashi T, Kohyama T, Wen F Q, Romberger D J, et al. Ultrafine carbon black particles inhibit human lung fibroblast-mediated collagen gel contraction. Am J Respir Cell Mol Biol. 2003, 28, 111-21.
[7] Cutroneo K R, White S L, Chiu J F, Ehrlich H P. Tissue fibrosis and carcinogenesis: divergent or successive pathways dictate multiple molecular therapeutic targets for oligo decoy therapies. J Cell Biochem. 2006, 97, 1161-74.
[8] Dobaczewski M, Gonzalez-Quesada C, Frangogiannis N G. The extracellular matrix as a modulator of the inflammatory and reparative response following myocardial infarction. J Mol Cell Cardiol. 2010, 48, 504-11.
[9] Kanzaki M, Yamato M, Takagi R, Kikkawa T, Isaka T, Okano T, et al. Controlled collagen crosslinking process in tissue-engineered fibroblast sheets for preventing scar contracture on the surface of lungs. J Tissue Eng Regen Med. 2012,
[10] Jackson W M, Nesti L J, Tuan R S. Mesenchymal stem cell therapy for attenuation of scar formation during wound healing. Stem Cell Res Ther. 2012, 3, 20.
[11] Yamaguchi Y, Kubo T, Murakami T, Takahashi M, Hakamata Y, Kobayashi E, et al. Bone marrow cells differentiate into wound myofibroblasts and accelerate the healing of wounds with exposed bones when combined with an occlusive dressing. Br J Dermatol. 2005, 152, 616-22.
[12] Hinz B, Gabbiani G. Fibrosis: recent advances in myofibroblast biology and new therapeutic perspectives. F1000 Biol Rep. 2010, 2, 78.
[13] Castella L F, Buscemi L, Godbout C, Meister J J, Hinz B. A new lock-step mechanism of matrix remodelling based on subcellular contractile events. J Cell Sci. 2010, 123, 1751-60.
[14] Gabbiani G. The myofibroblast in wound healing and fibrocontractive diseases. J Pathol. 2003, 200, 500-3.
[15] Hinz B. The myofibroblast: paradigm for a mechanically active cell. J Biomech. 2010, 43, 146-55.
[16] Liu Y. Cellular and molecular mechanisms of renal fibrosis. Nat Rev Nephrol. 2011, 7, 684-96.
[17] Schafer M, Werner S. Cancer as an overhealing wound: an old hypothesis revisited. Nat Rev Mol Cell Biol. 2008, 9, 628-38.
[18] Daskalopoulos E P, Janssen B J, Blankesteijn W M. Myofibroblasts in the infarct area: concepts and challenges. Microsc Microanal. 2012, 18, 35-49.
[19] Desmouliere A, Guyot C, Gabbiani G. The stroma reaction myofibroblast: a key player in the control of tumor cell behavior. Int J Dev Biol. 2004, 48, 509-17.
[20] Grinnell F. Fibroblast biology in three-dimensional collagen matrices. Trends Cell Biol. 2003, 13, 264-9.
[21] MacDonald R A, Voge C M, Kariolis M, Stegemann J P. Carbon nanotubes increase the electrical conductivity of fibroblast-seeded collagen hydrogels. Acta Biomater. 2008, 4, 1583-92.
[22] Dallon J C, Ehrlich H P. A review of fibroblast-populated collagen lattices. Wound Repair Regen. 2008, 16, 472-9.
[23] Sisco P N, Wilson C G, Mironova E, Baxter S C, Murphy C J, Goldsmith E C. The effect of gold nanorods on cell-mediated collagen remodeling. Nano Lett. 2008, 8, 3409-12.
[24] Supronowicz P R, Ajayan P M, Ullmann K R, Arulanandam B P, Metzger D W, Bizios R. Novel current-conducting composite substrates for exposing osteoblasts to alternating current stimulation. J Biomed Mater Res. 2002, 59, 499-506.
[25] Sohaebuddin S K, Thevenot P T, Baker D, Eaton J W, Tang L. Nanomaterial cytotoxicity is composition, size, and cell type dependent. Part Fibre Toxicol. 2010, 7, 22.
[26] Voge C M, Johns J, Raghavan M, Morris M D, Stegemann J P. Wrapping and dispersion of multiwalled carbon nanotubes improves electrical conductivity of protein-nanotube composite biomaterials. J Biomed Mater Res A. 2012, 101, 231-8.
[27] Wang X, Xia T, Ntim S A, Ji Z, Lin S, Meng H, et al. Dispersal state of multiwalled carbon nanotubes elicits profibrogenic cellular responses that correlate with fibrogcnesis biomarkers and fibrosis in the murine lung. ACS Nano. 2011, 5, 9772-87.
[28] Berlin J M, Leonard A D, Pham T T, Sano D, Marcano D C, Yan S, et al. Effective drug delivery, in vitro and in vivo, by carbon-based nanovectors noncovalently loaded with unmodified Paclitaxel. ACS Nano. 2010, 4, 4621-36.
[29] Schipper M L, Nakayama-Ratchford N, Davis C R, Kam N W, Chu P, Liu Z, et al. A pilot toxicology study of single-walled carbon nanotubes in a small sample of mice. Nat Nanotechnol. 2008, 3, 216-21.
[30] Bitner B R, Marcano D C, Berlin J M, Fabian R H, Cherian L, Culver J C, et al. Antioxidant carbon particles improve cerebrovascular dysfunction following traumatic brain injury. ACS Nano. 2012, 6, 8007-14.
[31] Kroll A, Dierker C, Rommel C, Hahn D, Wohileben W, Schulze-Isfort C, et al. Cytotoxicity screening of 23 engineered nanomaterials using a test matrix of ten cell lines and three different assays. Part Fibre Toxicol. 2011, 8, 9.
[32] Nel A E, Madler L, Velegol D, Xia T, Hoek E M, Somasundaran P, et al. Understanding biophysicochemical interactions at the nano-bio interface. Nat Mater. 2009, 8, 543-57.
[33] Berry C C, Shelton J C, Bader D L, Lee D A. Influence of external uniaxial cyclic strain on oriented fibroblast-seeded collagen gels. Tissue Eng. 2003, 9, 613-24.
[34] Marks M W, Morykwas M J, Wheatley M J. Fibroblast-mediated contraction in actinically exposed and actinically protected aging skin. Plast Reconstr Surg. 1990, 86, 255-9.
[35] Jing Li P C M, Wing Sze Chow, Chi Kai To, Ben Zhong Tang, Jang-Kyo Kim. Correlations between Percolation Threshold, Dispersion State, and Aspect Ratio of Carbon Nanotubes. Adv Funct Mater. 2007, 17, 3207-15.
[36] Bryan N, Ahswin H, Smart N, Bayon Y, Wohlert S, Hunt J A. Reactive oxygen species (ROS)—a family of fate deciding molecules pivotal in constructive inflammation and wound healing. Eur Cell Mater. 2012, 24, 249-65.
[37] Shi-wen X, Thompson K, Khan K, Liu S, Murphy-Marshman H, Baron M, et al. Focal adhesion kinase and reactive oxygen species contribute to the persistent fibrotic phenotype of lesional scleroderma fibroblasts. Rheumatology (Oxford). 2012, 51, 2146-54.
[38] Schlieve C R, Lieven C J, Levin L A. Biochemical activity of reactive oxygen species scavengers do not predict retinal ganglion cell survival. Invest Ophthalmol Vis Sci. 2006, 47, 3878-86.
[39] Gopalakrishnan R, Subramanian V. Interaction of collagen with carbon nanotube: a molecular dynamics investigation. J Biomed Nanotechnol. 2011, 7, 186-7.
[40] Park C O Z, Watson K A, Pawlowski S E, Lowther S E, Connell J W, et al. Polymer-single wall carbon nanotube composites for potential spacecraft applications. ICASE2002. 2002-36.
[41] Engler A J, Sen S, Sweeney H L, Discher D E. Matrix elasticity directs stem cell lineage specification. Cell. 2006, 126, 677-89.
[42] Zhang S, Zhu L, Wong C P, Kumar S. Polymer-Infiltrated Aligned Carbon Nanotube Fibers by in situ Polymerization. Macromol Rapid Commun. 2009, 30, 1936-9.
[43] Yan L Y, Chen H, Li P, Kim D H, Chan-Park M B. Finely dispersed single-walled carbon nanotubes for polysaccharide hydrogels. ACS Appl Mater Interfaces. 2012, 4, 4610-5.
[44] Xue P, Bao Y, Li Q, Wu C. Impact of modification of carbon black on morphology and performance of polyimide/carbon black hybrid composites. Phys Chem Chem Phys. 2010, 12, 11342-50.
[45] Munson-McGee S H. Estimation of the critical concentration in an anisotropic percolation network. Phys Rev B Condens Matter. 1991, 43, 3331-6.
[46] Shi G, Zhang Z, Rouabhia M. The regulation of cell functions electrically using biodegradable polypyrrole-polylactide conductors. Biomaterials. 2008, 29, 3792-8.

The foregoing is illustrative of the present invention, and is not to be construed as limiting thereof. The invention is defined by the following claims, with equivalents of the claims to be included therein.

1. A biomedical implant, comprising:
(a) an inert substrate;
(b) a coating on said substrate; and
(c) anisotropic nanoparticles in or on said coating in an amount effective to inhibit fibrosis, scarring, and/or fibrotic contracture of a tissue contacting said implant when implanted adjacent said tissue in a subject in need thereof;
wherein said nanoparticles are multi-wall carbon nanotubes.

2-3. (canceled)

4. The implant of claim 1, wherein said nanoparticles contact one another in sufficient number to form a network or lattice.

5. The implant of claim 1, wherein said implant comprises a breast implant or tissue expander.

6. The implant of claim 1, wherein said substrate comprises a hollow tube or a solid substrate.

7. The implant of claim 1, wherein said inert substrate comprises a flexible organic polymer.

8. The implant of claim 1, wherein said inert substrate comprises a rigid metal, metal oxide, carbon fiber, ceramic, organic polymer, or composite thereof.

9. The implant of claim 1, wherein said coating comprises a stable or biodegradable, natural or synthetic, porous or nonporous, organic polymer having an average thickness of from about 50 nanometers to 5 millimeters.

10. A method of inhibiting fibrosis, scarring, and/or fibrotic contracture of tissue contacting a biomedical implant in a patient implanted with said biomedical implant, comprising:
administering anisotropic nanoparticles to said tissue in an amount effective to inhibit fibrosis, scarring, and/or fibrotic contracture of said tissue;
wherein said nanoparticles are multi-wall carbon nanotubes.

11-12. (canceled)

13. The method of claim 10, wherein said nanoparticles contact one another in sufficient number to form a network or lattice.

14. The method of claim 10, wherein said subject is administered antineoplastic chemotherapy or radiotherapy prior to or after receiving said biomedical implant.

15. The method of claim 10, wherein said implant comprises a breast implant or tissue expander.

16. The method of claim 10, wherein said substrate comprises a hollow tube or solid substrate.

17. The method of claim 10, wherein said inert substrate comprises a flexible organic polymer.

18. The method of claim 10, wherein said inert substrate comprises a rigid metal, metal oxide, carbon fiber, ceramic, organic polymer, or composite thereof.

19. The method of claim 10, wherein said administering step is carried out by providing a coating on said implant, with said nanoparticles in or on said coating.

20. A regenerative template for implantation in a subject, comprising:
(a) a porous tissue scaffold; and
(b) anisotropic nanoparticles on said scaffold in an amount effective to inhibit fibrosis, scarring, and/or fibrotic contracture of tissue contacting said template when implanted in a subject in need thereof.

21-28. (canceled)

29. A method of inhibiting fibrosis, scarring, and/or fibrotic contracture of tissue contacting or infiltrating a regenerative template in a patient implanted with said regenerative template, comprising:
administering anisotropic nanoparticles to said tissue in an amount effective to inhibit fibrosis, scarring, and/or fibrotic contracture of said tissue.

30-37. (canceled)

38. A composition useful for inhibiting the formation of tissue adhesions in a subject in need thereof, comprising:
(a) a sterile pharmaceutically acceptable carrier, and
(b) anisotropic nanoparticles in said carrier.

39-42. (canceled)

43. A method of inhibiting the formation of tissue adhesions in a subject in need thereof, comprising topically administering anisotropic nanoparticles to said tissue in an effective adhesion-inhibiting amount.

44-49. (canceled)

50. An implant of claim 1, wherein said coating comprises a water binding polymer.

51-55. (canceled)
